(19) United States
(12) Patent Application Publication
(10) Pub. No.: US 2026/0053398 A1
(43) Pub. Date: Feb. 26, 2026

(54) BLOOD GLUCOSE METER AND METHOD FOR MEASURING BLOOD GLUCOSE

(71) Applicant: National Central University, Taoyuan City (TW)

(72) Inventor: Po-Lei LEE, Taoyuan City (TW)

(21) Appl. No.: 18/812,891

(22) Filed: Aug. 22, 2024

(30) Foreign Application Priority Data
Aug. 21, 2024 (TW) 113131401

(51) Int. Cl. A61B 5/1455; A61B 5/024; A61B 5/145

(52) U.S. Cl. CPC A61B 5/1455; A61B 5/14532; A61B 5/02433

(57) ABSTRACT

A blood glucose meter is provided. The blood glucose meter mainly includes a first light source, a second light source, a light sensor, a PPG circuit and a processing unit. The first light source and the second light source respectively emit a first light energy and a second light energy to human skin. The light sensor is used to convert the first light energy and the second light energy reflected off human skin or passing through human skin into a first electrical signal and a second electrical signal. The PPG circuit is configured to generate a first PPG response and a second PPG response according to the first electrical signal and the second electrical signal. A processing unit is configured to execute the following steps: performing feature extraction according to the first PPG response and the second PPG response to obtain a first set of feature values and a second set of feature values; performing an algorithm to obtain a first group of feature ratio values according to the first set of feature values and the second set of feature values; and applying the first group of feature ratio values to a regression model to obtain a blood glucose value.

### CROSS REFERENCE TO RELATED APPLICATION

This application claims the priority of Taiwan Patent Application No. 113131401, filed on Aug. 22, 2024, entitled “Blood glucose meter and method for measuring blood glucose”, and the disclosure of which is incorporated herein by reference.

### FIELD OF INVENTION

The present disclosure relates to a health data sensor, and more particularly, to a non-invasive blood glucose meter that uses multiple light sources to detect blood glucose.

### BACKGROUND OF INVENTION

Current blood glucose meters are categorized into invasive and non-invasive types. Invasive blood glucose meters have been developed for many years and are relatively mature in technology, with the advantage of high accuracy. However, they require pricking the skin to obtain a blood sample, causing discomfort and pain to the patient. The risk of infection is particularly heightened with frequent measurements. Additionally, each measurement requires a certain amount of time and operational steps, significantly increasing the cost for patients who need frequent monitoring.

Non-invasive blood glucose meters do not require pricking the skin but are implemented through optical measurement, electromagnetic field measurement, ultrasound measurement, and skin impedance measurement. Non-invasive blood glucose meters are easy to operate, but individual differences between users, along with technical differences in data analysis across different brands, leading to measurement errors in blood glucose and affect data accuracy. As a result, their precision is generally lower than that of invasive blood glucose meters. This is especially true at the extreme ends of the glucose measurement range, where frequent calibration is needed to ensure data accuracy. Furthermore, the measurement results may be affected by external environmental factors, such as temperature, humidity, light, etc. Therefore, it is necessary to develop more effective technologies to improve the accuracy and environmental sensitivity of non-invasive blood glucose meters.

Currently, patents related to non-invasive blood glucose meters include Taiwan Patent No. TWI555510B, entitled “Non-invasive blood glucose measurement device and measurement method using the same,” Taiwan Patent No. TWI385387B, entitled “Non-invasive blood glucose meter with array electrodes,” and U.S. Pat. No. 9,642,578B2, entitled “System and method for health monitoring using a non-invasive, multi-band biosensor.”

### SUMMARY OF INVENTION

In view of the shortcomings of the prior art mentioned above, the present disclosure proposes a blood glucose meter that utilizes at least two different wavelengths of light sources to measure blood glucose and employs a specific method to analyze the corresponding PPG (Photoplethysmography) signal responses to obtain highly accurate blood glucose values.

The present disclosure provides a blood glucose meter, comprising: a first light source, having a first wavelength, configured to: emit a first light energy towards human skin; a second light source, having a second wavelength, configured to: be positioned on the same side as the first light source and emit a second light energy towards human skin, wherein the second wavelength is greater than the first wavelength; a light sensor, configured to: be positioned on the opposite side of the first light source to convert the first light energy and the second light energy that have passed through human skin into a first electrical signal and a second electrical signal, respectively; or be positioned on the same side as the first light source to convert the first light energy and the second light energy reflected off human skin into the first electrical signal and the second electrical signal, respectively; a PPG circuit, configured to: drive the first light source and the second light source, and generate a first PPG response and a second PPG response based on the first electrical signal and the second electrical signal; a processing unit, configured to execute the following steps: performing feature extraction based on the first PPG response and the second PPG response to obtain a first set of feature values and a second set of feature values; executing an algorithm based on the first set of feature values and the second set of feature values to obtain a first group of feature ratio values; and applying the first group of feature ratio values to a regression model to obtain a blood glucose value; and a display module, configured to: display the blood glucose value.

In one embodiment of the present disclosure, the first group of feature ratio values includes a first subgroup of feature ratio values and a second subgroup of feature ratio values, and the algorithm includes the following operations: performing a difference operation on the first set of feature values and the second set of feature values to obtain a first set of difference values and a second set of difference values; performing logarithmic, division, and subtraction operations on the first set of difference values and the second set of difference values to obtain the first subgroup of feature ratio values; and performing a division operation on the first set of difference values and the second set of difference values to obtain the second subgroup of feature ratio values.

In one embodiment of the present disclosure, further comprising a third light source having a third wavelength, configured to: be positioned on the same side as the first light source and emit a third light energy towards human skin, wherein the third wavelength is greater than the first wavelength, and the light sensor is further configured to convert the third light energy that passed through human skin into a third electrical signal; or convert the third light energy reflected off human skin into the third electrical signal, wherein the PPG circuit is further configured to: drive the third light source and generate a third PPG response based on the third electrical signal, wherein, the processing unit is further configured to execute the following steps: performing feature extraction based on the third PPG response to obtain a third set of feature values; executing the algorithm based on the first set of feature values and the third set of feature values to obtain a second group of feature ratio values; and applying the first group of feature ratio values and the second group of feature ratio values to the regression model to obtain the updated blood glucose value.

In one embodiment of the present disclosure, the second group of feature ratio values includes a third subgroup of feature ratio values and a fourth subgroup of feature ratio values, and the algorithm further includes the following operations: performing a difference operation on the third set of feature values to obtain a third set of difference values; performing logarithmic, division, and subtraction operations on the first set of difference values and the third set of difference values to obtain the third subgroup of feature ratio values; and performing a division operation on the first set of difference values and the third set of difference values to obtain the fourth subgroup of feature ratio values.

In one embodiment of the present disclosure, the first light source is green light, the second light source is red light, and the third light source is near-infrared light.

The present disclosure provides a method for measuring blood glucose, comprising: driving a first light source having a first wavelength and a second light source having a second wavelength by a PPG circuit, such that the first light source emits a first light energy towards human skin and the second light source emits a second light energy towards human skin, wherein the second wavelength is greater than the first wavelength; converting the first light energy and the second light energy that have passed through human skin into a first electrical signal and a second electrical signal respectively, or converting the first light energy and the second light energy reflected off human skin into the first electrical signal and the second electrical signal respectively by a light sensor; generating a first PPG response and a second PPG response based on the first electrical signal and the second electrical signal by the PPG circuit; executing the following steps by a processing unit: performing feature extraction based on the first PPG response and the second PPG response to obtain a first set of feature values and a second set of feature values; executing an algorithm based on the first set of feature values and the second set of feature values to obtain a first group of feature ratio values; and applying the first group of feature ratio values to a regression model to obtain a blood glucose value; and displaying the blood glucose value by a display module.

In one embodiment of the present disclosure, the first group of feature ratio values includes a first subgroup of feature ratio values and a second subgroup of feature ratio values, and the algorithm includes the following operations: performing a difference operation on the first set of feature values and the second set of feature values to obtain a first set of difference values and a second set of difference values; performing logarithmic, division, and subtraction operations on the first set of difference values and the second set of difference values to obtain the first subgroup of feature ratio values; and performing a division operation on the first set of difference values and the second set of difference values to obtain the second subgroup of feature ratio values.

In one embodiment of the present disclosure, further comprising: driving a third light source by the PPG circuit, such that the third light source emits a third light energy with a third wavelength towards human skin, wherein the third wavelength is greater than the first wavelength; converting the third light energy that passed through human skin into a third electrical signal, or converting the third light energy reflected off human skin into the third electrical signal by the light sensor. generating a third PPG response based on the third electrical signal by the PPG circuit; and executing the following steps by the processing unit: performing feature extraction based on the third PPG response to obtain a third set of feature values; executing the algorithm based on the first set of feature values and the third set of feature values to obtain a second group of feature ratio values; and applying the first group of feature ratio values and the second group of feature ratio values to the regression model to obtain the updated blood glucose value.

In one embodiment of the present disclosure, the second group of feature ratio values includes a third subgroup of feature ratio values and a fourth subgroup of feature ratio values, and the algorithm further includes the following operations: performing a difference operation on the third set of feature values to obtain a third set of difference values; performing logarithmic, division, and subtraction operations on the first set of difference values and the third set of difference values to obtain the third subgroup of feature ratio values; and performing a division operation on the first set of difference values and the third set of difference values to obtain the fourth subgroup of feature ratio values.

In one embodiment of the present disclosure, further comprising: driving a nth light source by the PPG circuit, such that the nth light source emits an nth light source towards human skin, where the wavelength of the nth light source is greater than the first wavelength, and n>3; converting the nth light energy that passed through the human skin into the nth electrical signal, or converting the nth light energy reflected off the human skin into the nth electrical signal by the light sensor; generating an nth PPG response based on the nth electrical signal by the PPG circuit; and executing the following steps by the processing unit: performing feature extraction based on the nth PPG response to obtain the nth set of feature values; performing a difference operation on the nth set of feature values to obtain the nth set of difference values; performing logarithmic, division, and subtraction operations on the first set of difference values and the nth set of difference values to obtain a ((n−1)×2−1)th subgroup of feature ratio values; performing a division operation on the first set of difference values and the nth set of difference values to obtain a ((n−1)×2)th subgroup of feature ratio values; and applying the first subgroup of feature ratio values to the ((n−1)×2)th subgroup feature ratio values to the regression model to obtain the updated blood glucose value.

### DESCRIPTION OF DRAWINGS

FIG. 1 shows a schematic diagram of the structure of a blood glucose meter according to the present disclosure.

FIG. 2 shows a schematic diagram of multiple PPG responses obtained by a PPG circuit of the blood glucose meter according to the present disclosure.

FIG. 3 shows a schematic diagram of a single PPG response from FIG. 2.

FIG. 4 shows a flowchart of the method for measuring blood glucose according to the present disclosure.

### DETAILED DESCRIPTION OF PREFERRED EMBODIMENTS

In order to make the above and other objectives, features, and advantages of the present disclosure more obvious and understandable, the following exemplifies the preferred embodiments of the present disclosure, combined with the accompanying drawings, and describe in detail as follows.

It should be understood that the following detailed description of the embodiments is merely illustrative and should not be construed as limiting the invention as defined by the claims. In general, unless the following embodiments explicitly define the terms used, the terms included in the disclosure should not be interpreted as limiting the techniques described herein to the specific embodiments disclosed in this specification. Accordingly, the actual scope of the techniques described herein should encompass the disclosed embodiments as well as all equivalent methods for implementing these techniques.

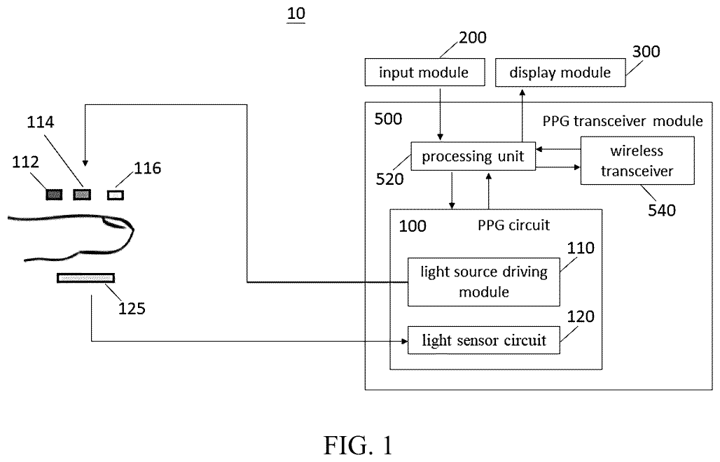

Firstly, refer to FIG. 1. FIG. 1 shows a schematic diagram of the structure of a blood glucose meter according to the present disclosure. The blood glucose meter 10 of the present disclosure includes: a first light source 112, a second light source 114, a light sensor 125, an input module 200, a display module 300, and a PPG transceiver module 500. In an embodiment of the present disclosure, the blood glucose meter 10 may optionally include a third light source 116. In other embodiments, the blood glucose meter 10 may include more light sources (not shown).

The input module 200 may be a keyboard, a touch panel, buttons, or the like. The display module 300 may be a liquid crystal display (LCD), an OLED display, or similar devices. The first light source 112, the second light source 114, and the third light source 116 can be light-emitting diodes (LEDs) or lasers, among others. The light sensor 125 can be a photodiode, and is not limited thereto.

In one embodiment, the first light source 112 emits green light with a wavelength of approximately 530 nm; the second light source 114 emits red light with a wavelength of approximately 780 nm; and the third light source 116 emits near-infrared light with a wavelength of approximately 830 nm. The first light source 112, the second light source 114, and the third light source 116 are all positioned on the same side. A first light energy generated by the first light source 112, a second light energy generated by the second light source 114, and a third light energy generated by the third light source 116 are all configured to be emitted towards human skin, typically towards the skin of a finger.

The light sensor 125 can be positioned on the opposite side of the first light source 112, the second light source 114, and the third light source 116, or on the same side as these light sources. When the light sensor 125 is positioned on the opposite side, it converts the first light energy, the second light energy, and the third light energy that passed through human skin into the first electrical signal, the second electrical signal, and the third electrical signal, respectively. When the light sensor 125 is positioned on the same side as the light sources, it converts the light energies reflected off human skin into the electrical signals.

The PPG transceiver module 500 includes a PPG circuit 100, a processing unit 520, and a wireless transceiver 540. A light source driving module 110 of the PPG circuit 100 is configured to drive the first light source 112, the second light source 114, and the third light source 116. A light sensor circuit 120 of the PPG circuit 100 is configured to generate the first PPG response, the second PPG response, and the third PPG response based on the first electrical signal, the second electrical signal, and the third electrical signal, respectively. The processing unit 520 may include a microprocessor, microchip, integrated circuit, or similar components for processing data and/or executing algorithms and various calculations as described below. The wireless transceiver 540 may include a Bluetooth transceiver, a WiFi transceiver, or other transceivers compatible with wireless standards, such as RFID and short-range communication, among others.

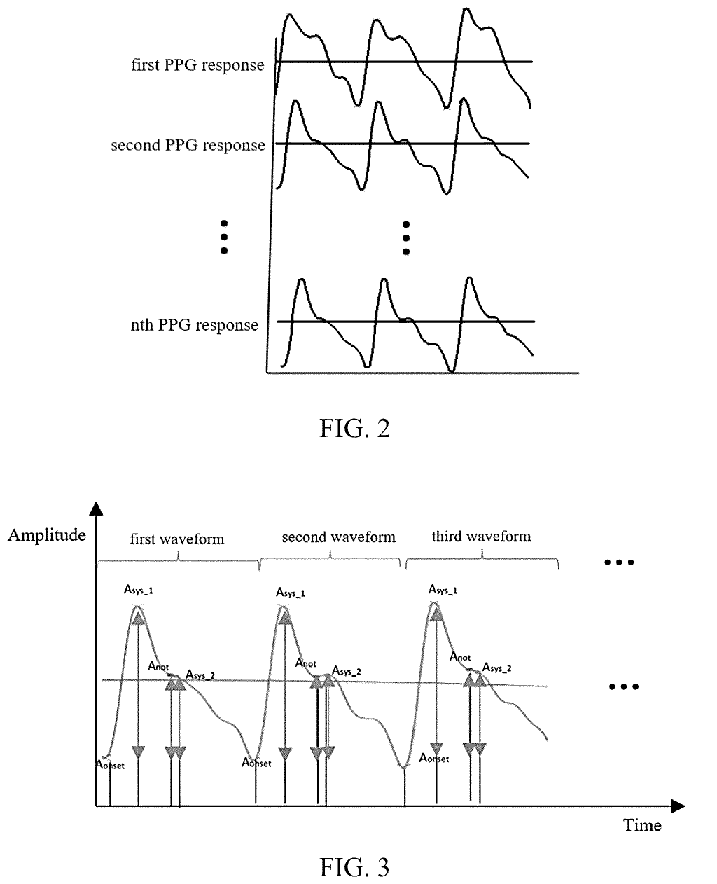

Please refer to FIG. 2 and FIG. 3. FIG. 2 shows a schematic diagram of multiple PPG responses obtained by the PPG circuit of the blood glucose meter according to the present disclosure. FIG. 3 shows a schematic diagram of a single PPG response from FIG. 2. Although this description only explains the use of two or three PPG responses to estimate blood glucose values, in other embodiments, more PPG responses generated by additional light sources can be used to estimate blood glucose values. Each PPG response includes multiple waveforms. Each waveform has similar feature points, including the onset, systolic peak, dicrotic notch, and diastolic peak. In FIG. 3, Aₒₙₛₑₜ represents the amplitude of the onset for each waveform, A_{sys_1} represents the amplitude of the systolic peak for each waveform, Aₙₒₜ represents the amplitude of the dicrotic notch for each waveform, and A_{sys_2} represents the amplitude of the diastolic peak for each waveform.

In one embodiment, the blood glucose meter 10 is configured with only the first light source 112 and the second light source 114, and the PPG circuit 100 obtains the first PPG response and the second PPG response, respectively. The processing unit 520 then performs feature extraction based on the first PPG response to obtain the first set of feature values and based on the second PPG response to obtain the second set of feature values. The first set of feature values refers to the collection of amplitudes of the feature points of the first PPG response. The second set of feature values refers to the collection of amplitudes of the feature points of the second PPG response.

Please refer to FIG. 3 again. When the PPG response in FIG. 3 represents the first PPG response (that is, the PPG generated by the first electrical signal corresponding to the first light energy emitted by the first light source 112 with a first wavelength λ1 after it is reflected off or passes through the skin), the first set of feature values includes the amplitudes of the feature points from multiple waveforms of the first PPG response. The amplitude at each waveform's feature point is denoted as A_{onset(λ1)} for the onset amplitude, A_{sys_1(λ1)} for the systolic peak amplitude, A_{not(λ1)} for the dicrotic notch amplitude, and A_{sys_2(λ1)} for the diastolic peak amplitude. Similarly, when the PPG response in FIG. 3 represents the second PPG response, that is, the PPG generated by the second electrical signal corresponding to the second light energy emitted by the second light source 114 with a second wavelength λ2 after it is reflected off or passes through the skin, the second set of feature values includes the amplitudes of the feature points from multiple waveforms of the second PPG response. The amplitude at each waveform's feature point is denoted as A_{onset(λ2)}, A_{sys_1(λ2)}, A_{not(λ2)}, and A_{sys_2(λ2)}. Likewise, when the PPG response in FIG. 3 represents the nth PPG response, that is, when there are n light sources, the amplitude at each waveform's feature point is denoted as A_{onset(λn)}, A_{sys_1(λn)}, A_{not(λn)}, and A_{sys_2(λn)}.

After obtaining the first set of feature values and the second set of feature values, the processing unit 520 further calculates the difference values between the feature values according to each set of feature values. In this embodiment, it is focused on calculating the difference values between the amplitudes of the systolic peak, dicrotic notch, and diastolic peak and the amplitude of the onset. Taking FIG. 3 as an example, the difference value between the systolic peak amplitude A_{sys_1} and the onset amplitude Aₒₙₛₑₜ is denoted as Aₛ₁₋ₒ, i.e., Aₛ₁₋ₒ=A_{sys_1}−Aₒₙₛₑₜ. The difference value between the dicrotic notch amplitude Aₙₒₜ and the onset amplitude Aₒₙₛₑₜ is denoted as Aₙ₋ₒ, i.e., Aₙ₋ₒ=Aₙₒₜ−Aₒₙₛₑₜ. The difference value between the diastolic peak amplitude A_{sys_2} and the onset amplitude Aₒₙₛₑₜ is denoted as Aₛ₂₋ₒ, i.e., Aₛ₂₋ₒ=A_{sys_2}−Aₒₙₛₑₜ. Similarly, when FIG. 3 represents the first PPG response, the difference values between the first set of feature values are denoted as A_{s1-o(λ1)}, A_{n-0(λ1)}, and A_{s2-o(λ1)} (hereinafter referred to as the first set of difference values). When FIG. 3 represents the second PPG response, the difference values between the second set of feature values are denoted as A_{s1-o(λ2)}, A_{n-o(λ2)}, and A_{s2-o(λ2)} (hereinafter referred to as the second set of difference values). Likewise, when FIG. 3 represents the nth PPG response, the difference values between the nth feature values are denoted as A_{s1-o(λn)}, A_{n-o(λn)}, and A_{s2-o(λn)} (hereinafter referred to as the nth set of difference values).

After calculating the difference values between each set of feature values, the processing unit 520 will perform calculations based on the first set of difference values and the second set of difference values to obtain two subgroup feature ratio values (a total of six feature ratio values). These six feature ratio values are defined as follows:

$${R}_{s 1 - o - 1} = \frac{log ( {A}_{s 1 - o ( λ n )} )}{log ( {A}_{s 1 - o ( λ 1 )} )} - K \qquad ( equation 1 - 1 )$$

$${R}_{n - o - 1} = \frac{log ( {A}_{n - o ( λ n )} )}{log ( {A}_{n - o ( λ 1 )} )} - K \qquad ( equation 1 - 2 )$$

$${R}_{s 2 - o - 1} = \frac{log ( {A}_{s 2 - o ( λ n )} )}{log ( {A}_{s 2 - o ( λ 1 )} )} - K \qquad ( equation 1 - 3 )$$

$${R}_{s 1 - o - 2} = \frac{{A}_{s 1 - o ( λ n )}}{{A}_{s 1 - o ( λ 1 )}} \qquad ( equation 2 - 1 )$$

$${R}_{n - o - 2} = \frac{{A}_{s 1 - o ( λ n )}}{{A}_{s 1 - o ( λ 1 )}} \qquad ( equation 2 - 2 )$$

$${R}_{s 2 - o - 2} = \frac{{A}_{s 1 - o ( λ n )}}{{A}_{s 1 - o ( λ 1 )}} \qquad ( equation 2 - 3 )$$

where K is a constant in the regression curve.

where n>1, and n equals to the number of light sources. In this embodiment, the number of light sources is at least two.

For example, in an embodiment where the number of light sources is two, after the processing unit 520 calculates the difference values between the first set of feature values and the second set of feature values, it will use the above equations 1-1 to 1-3 to obtain the following one subgroup of feature ratio values (three feature ratio values) based on the first set of difference values and the second set of difference values.

$${R}_{s 1 - o - 1} = \frac{log ( {A}_{s 1 - o ( λ 2 )} )}{log ( {A}_{s 1 - o ( λ 1 )} )} - K$$

$${R}_{n - o - 1} = \frac{log ( {A}_{n - o ( λ 2 )} )}{log ( {A}_{n - o ( λ 1 )} )} - K$$

$${R}_{s 2 - o - 1} = \frac{log ( {A}_{s 2 - o ( λ 2 )} )}{log ( {A}_{s 2 - o ( λ 1 )} )} - K$$

Additionally, the processing unit 520 will use the above equations 2-1 to 2-3 to obtain another subgroup of feature ratio values (also three feature ratio values) based on the first set of difference values and the second set of difference values.

$${R}_{s 1 - o - 2} = \frac{{A}_{s 1 - o ( λ 2 )}}{{A}_{s 1 - o ( λ 1 )}}$$

$${R}_{n - o - 2} = \frac{{A}_{s 1 - o ( λ 2 )}}{{A}_{s 1 - o ( λ 1 )}}$$

$${R}_{s 2 - o - 2} = \frac{{A}_{s 1 - o ( λ 2 )}}{{A}_{s 1 - o ( λ 1 )}}$$

After calculating the six feature ratio values, the processing unit 520 applies these feature ratio values to a regression model to finally obtain the blood glucose value, which is then displayed on the display module 300. In this embodiment, the regression model may include linear regression, support vector regression, and random forest regression, and is not limited thereto.

The establishment and training of the regression model involve collecting large datasets for machine learning. This is achieved by using PPG signals and collected blood glucose values as inputs to train the feature extraction and R-value calculations mentioned above, resulting in the regression model relating blood glucose values to PPG signals. Subsequently, when a user operates the blood glucose meter 10, the processing unit 520 can apply the current PPG responses to the regression model to determine the user's blood glucose value.

In another embodiment, when the blood glucose meter 10 includes the third light source 116, the PPG circuit 100 not only acquires the first and second PPG responses but also acquires a third PPG response. The processing unit 520, in addition to performing feature extraction based on the first PPG response to obtain the first set of feature values and based on the second PPG response to obtain the second set of feature values, will also perform feature extraction based on the third PPG response to obtain the third set of feature values.

Please refer to FIG. 3 again. When the PPG response in FIG. 3 represents the third PPG response (i.e., the PPG response generated by a third electrical signal corresponding to a third light energy that is reflected off or passes through the skin, after the third light source 116 with a third wavelength λ3 emits the skin), the third set of feature values includes the amplitudes of each feature point from the multiple waveforms of the third PPG response. The amplitudes of the feature points for each waveform are denoted as A_{onset(λ3)}, A_{sys_1(λ3)}, A_{not(λ3)}, and A_{sys_2(λ3)}.

After obtaining the first set of feature values to the third set of feature values, the processing unit 520 will calculate the difference values between feature values according to each set of feature values. The calculation of difference values between the first set feature values and the second set of feature values has been described earlier and will not be repeated here. Referring to FIG. 3, when FIG. 3 represents the third PPG response, the difference values between the third set of feature values are denoted as: A_{s1-o(λ3)}, A_{n-o(λ3)}, and A_{s2-o(λ3)} (hereinafter referred to as the third set of difference values).

In an embodiment with three light sources, after calculating the difference values between each set of feature values, the processing unit 520 will use these difference values to obtain two groups of feature ratio values (a total of twelve feature ratio values) based on the above equations. As previously mentioned, the processing unit 520 will obtain two subgroup of feature ratio values (a total of six feature ratio values) based on the first set of difference values and the second set of difference values using the above equations. Additionally, the processing unit 520 will use the first set of difference values and the third set of difference values to obtain the following two additional subgroup of feature ratio values (also six feature ratio values) using the above equations, as follows:

$${R}_{s 1 - o - 1} = \frac{log ( {A}_{s 1 - o ( λ 3 )} )}{log ( {A}_{s 1 - o ( λ 1 )} )} - K$$

$${R}_{n - o - 1} = \frac{log ( {A}_{n - o ( λ 3 )} )}{log ( {A}_{n - o ( λ 1 )} )} - K$$

$${R}_{s 2 - o - 1} = \frac{log ( {A}_{s 2 - o ( λ 3 )} )}{log ( {A}_{s 2 - o ( λ 1 )} )} - K$$

$${R}_{s 1 - o - 2} = \frac{{A}_{s 1 - o ( λ 3 )}}{{A}_{s 1 - o ( λ 1 )}}$$

$${R}_{n - o - 2} = \frac{{A}_{s 1 - o ( λ 3 )}}{{A}_{s 1 - o ( λ 1 )}}$$

$${R}_{s 2 - o - 2} = \frac{{A}_{s 1 - o ( λ 3 )}}{{A}_{s 1 - o ( λ 1 )}}$$

After calculating all the feature ratio values, the processing unit 520 applies the feature ratio values (a total of twelve feature ratio values) to the regression model, obtaining a blood glucose value that may differ from the one obtained by applying only six feature ratio values.

Similarly, if the blood glucose meter 10 is equipped with a fourth light source, a fifth light source, and up to a nth light source, the processing unit 520 will calculate the first set of difference values to the nth set of difference values. Then, the processing unit 520 will use the above six equations to obtain six feature ratio values based on the first set of difference values and the second set of difference values, to obtain another six feature ratio values based on the first set of difference values and the third set of difference values, and so on, to again obtain six feature ratio values, that is, the (n−1)th group of feature ratio values, which includes the ((n−1)×2−1)th subgroup of feature ratio values and the ((n−1)×2)th subgroup of feature ratio values, based on the first difference values and the nth set of difference values. All these feature ratio values, a total of 6×(n−1) feature ratio values, are then applied to the regression model to obtain the blood glucose value.

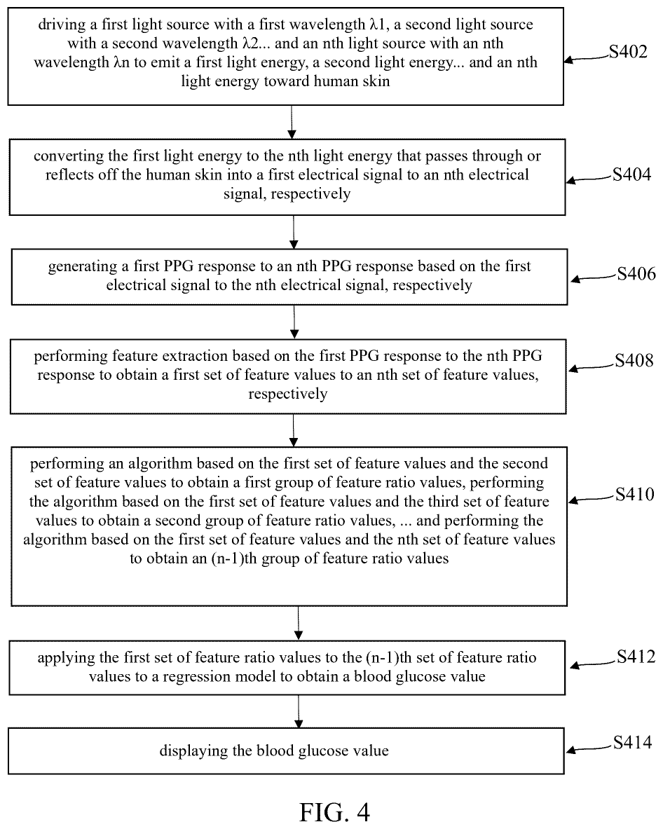

Please refer to FIG. 4. FIG. 4 shows a flowchart of the method for measuring blood glucose according to the present disclosure. First, in Step S402: driving a first light source with a first wavelength λ1, a second light source with a second wavelength λ2 . . . and an nth light source with an nth wavelength λn to emit a first light energy, a second light energy . . . and an nth light energy toward human skin. In one embodiment, the blood glucose meter 10 is equipped with only two light sources. In another embodiment, the blood glucose meter 10 is equipped with three light sources. Preferably, the first light source 112 is green light with a wavelength of approximately 530 nm; the second light source 114 is red light with a wavelength of approximately 780 nm; and the third light source 116 is near-infrared light with a wavelength of approximately 830 nm. The first light source 112, second light source 114, and third light source 116 are all arranged on the same side. The first light energy generated by the first light source 112, the second light energy generated by the second light source 114, and the third light energy generated by the third light source 116 are all configured to be emitted toward human skin, typically toward the skin of a finger.

Step S404: converting the first light energy to the nth light energy that passes through or reflects off the human skin into a first electrical signal to an nth electrical signal, respectively. In one embodiment, when the light sensor 125 is placed on the opposite side of the light sources, it converts the first light energy, second light energy . . . and nth light energy that passes through the human skin into the first electrical signal, second electrical signal . . . and nth electrical signal, respectively. In another embodiment, when the light sensor 125 is placed on the same side as the light sources, it converts the light energy reflected off the human skin into the corresponding electrical signals.

Step S406: generating a first PPG response to an nth PPG response based on the first electrical signal to the nth electrical signal, respectively. The PPG circuit 100 is configured to generate the first PPG response, second PPG response . . . and nth PPG response based on the first electrical signal, second electrical signal . . . and nth electrical signal.

Step S408: performing feature extraction based on the first PPG response to the nth PPG response to obtain a first set of feature values to an nth set of feature values, respectively. The feature points at each waveform of each PPG response include the onset, systolic peak, dicrotic notch, and diastolic peak. The feature extraction herein refers to extracting the amplitude of the feature points at a waveform of a PPG response. The amplitude of the onset point herein is denoted as Aₒₙₛₑₜ, the amplitude of the systolic peak is denoted as A_{sys_1}, the amplitude of the dicrotic notch is denoted as Aₙₒₜ, and the amplitude of the diastolic peak is denoted as A_{sys_2}.

In one embodiment, the blood glucose meter 10 is equipped with two light sources, and the PPG circuit generates two PPG responses (referred to as the first PPG response and the second PPG response). The first set of feature values refers to a set of amplitudes of the feature points from the multiple waveforms of the first PPG response. The second set of feature values refers to a set of amplitudes of the feature points from the multiple waveforms of the second PPG response. Therefore, the feature values of each waveform in the first PPG response are labeled as A_{onset(λ1)}, A_{sys_1(λ1)}, A_{not(λ1)}, and A_{sys_2(λ1)}. Similarly, the feature values of each waveform in the second PPG response are labeled as A_{onset(λ2)}, A_{sys_1(λ2)}, A_{not(λ2)}, and A_{sys_2(λ2)}. In another embodiment, if the blood glucose meter 10 is equipped with three light sources, the PPG circuit generates three PPG responses (hereinafter referred to as the first PPG response, the second PPG response, and the third PPG response). The feature values of each waveform in the third PPG response are labeled as A_{onset(λ3)}, A_{sys_1(λ3)}, A_{not(λ3)}, and A_{sys_2(λ3)}. Similarly, if the blood glucose meter 10 is equipped with n light sources, the PPG circuit generates n PPG responses, and the feature values of each waveform in the nth PPG response are labeled as A_{onset(λn)}, A_{sys_1(λn)}, A_{not(λn)}, and A_{sys_2(λn)}.

Step S410: performing an algorithm based on the first set of feature values and the second set of feature values to obtain a first group of feature ratio values, performing the algorithm based on the first set of feature values and the third set of feature values to obtain a second group of feature ratio values, . . . and performing the algorithm based on the first set of feature values and the nth set of feature values to obtain an (n−1)th group of feature ratio values.

The feature ratio values herein are defined as follows:

$${R}_{s 1 - o - 1} = \frac{log ( {A}_{s 1 - o ( λ n )} )}{log ( {A}_{s 1 - o ( λ 1 )} )} - K \qquad ( equation 1 - 1 )$$

$${R}_{n - o - 1} = \frac{log ( {A}_{n - o ( λ n )} )}{log ( {A}_{n - o ( λ 1 )} )} - K \qquad ( equation 1 - 2 )$$

$${R}_{s 2 - o - 1} = \frac{log ( {A}_{s 2 - o ( λ n )} )}{log ( {A}_{s 2 - o ( λ 1 )} )} - K \qquad ( equation 1 - 3 )$$

$${R}_{s 1 - o - 2} = \frac{{A}_{s 1 - o ( λ n )}}{{A}_{s 1 - o ( λ 1 )}} \qquad ( equation 2 - 1 )$$

$${R}_{n - o - 2} = \frac{{A}_{s 1 - o ( λ n )}}{{A}_{s 1 - o ( λ 1 )}} \qquad ( equation 2 - 2 )$$

$${R}_{s 2 - o - 2} = \frac{{A}_{s 1 - o ( λ n )}}{{A}_{s 1 - o ( λ 1 )}} \qquad ( equation 2 - 3 )$$

Where K is a constant in the regression curve.

Where n>1, n equals to the number of light sources.

Before calculating the feature ratio values, that is, after obtaining each set of feature values, the processing unit 520 must also calculate the difference values between the feature values for each set of feature values. In this embodiment, it is focused on calculating the difference values between the amplitudes of the systolic peak, the dicrotic notch, and the diastolic peak and the amplitude of the onset point. Taking FIG. 3 as an example, the difference values between the amplitude of the systolic peak A_{sys_1} and the amplitude of the onset point Aₒₙₛₑₜ is Aₛ₁₋ₒ, i.e., Aₛ₁₋ₒ=A_{sys_1}−Aₒₙₛₑₜ. The difference values between the amplitude of the dicrotic notch Aₙₒₜ and the amplitude of the onset point Aₒₙₛₑₜ is Aₙ₋ₒ, i.e., Aₙ₋ₒ=Aₙₒₜ−Aₒₙₛₑₜ. The difference values between the amplitude of the diastolic peak A_{sys_2} and the amplitude of the onset point Aₒₙₛₑₜ is Aₛ₂₋ₒ, i.e., Aₛ₂₋ₒ=A_{sys_2}−Aₒₙₛₑₜ. Similarly, the difference values between the first set of feature values are A_{s1-o(λ1)}, A_{n-o(λ1)}, and A_{s2-o(λ1)} (hereinafter referred to as the first set of difference values); the difference values between the second set of feature values are A_{s1-o(λ2)}, A_{n-o(λ2)}, and A_{s2-o(λ2)} (hereinafter referred to as the second set of difference values); and the difference values between the third set of feature values are A_{s1-o(λ3)}, A_{n-o(λ3)}, and A_{s2-o(λ3)} (hereinafter referred to as the third set of difference values). Similarly, the difference values between the nth set of feature values are A_{s1-o(λn)}, A_{n-o(λn)}, and A_{s2-o(λn)} (hereinafter referred to as the nth set of difference values).

After calculating the difference values between each set of feature values, the processing unit 520 will obtain multiple subgroups of feature ratio values, that is, each subgroup includes three feature ratio values, according to the second set of difference values to the nth set of difference values and the first set of difference values using the aforementioned equations 1-1 to 1-3. For simplicity, only the second set of difference values and the first set of difference values are illustrated as an example, and the feature ratio values are as follows:

$${R}_{s 1 - o - 1} = \frac{log ( {A}_{s 1 - o ( λ 2 )} )}{log ( {A}_{s 1 - o ( λ 1 )} )} - K$$

$${R}_{n - o - 1} = \frac{log ( {A}_{n - o ( λ 2 )} )}{log ( {A}_{n - o ( λ 1 )} )} - K$$

$${R}_{s 2 - o - 1} = \frac{log ( {A}_{s 2 - o ( λ 2 )} )}{log ( {A}_{s 2 - o ( λ 1 )} )} - K$$

Additionally, the processing unit 520 will obtain another subgroup of feature ratio values according to the first set of difference values and the second set of difference values using the aforementioned equations 2-1 to 2-3.

$${R}_{s 1 - o - 2} = \frac{{A}_{s 1 - o ( λ 2 )}}{{A}_{s 1 - o ( λ 1 )}}$$

$${R}_{n - o - 2} = \frac{{A}_{s 1 - o ( λ 2 )}}{{A}_{s 1 - o ( λ 1 )}}$$

$${R}_{s 2 - o - 2} = \frac{{A}_{s 1 - o ( λ 2 )}}{{A}_{s 1 - o ( λ 1 )}}$$

In another embodiment, when a third PPG response is present, that is, when there is a third set of feature values, the processing unit 520, after obtaining the first set of feature values to the third set of feature values, not only calculates the difference values between the first set of feature values and the second set of feature values but also calculates the difference values between the first set of feature values and the third set of feature values.

As mentioned earlier, the processing unit 520 will obtain two subgroups of feature ratio values (a total of six feature ratio values) according to the first set of difference values and the second set of difference values using the aforementioned equations. In addition, the processing unit 520 will obtain the following additional two subgroups of feature ratio values (also six feature ratio values) according to the first set of difference values and the third set of difference values.

$${R}_{s 1 - o - 1} = \frac{log ( {A}_{s 1 - o ( λ 3 )} )}{log ( {A}_{s 1 - o ( λ 1 )} )} - K$$

$${R}_{n - o - 1} = \frac{log ( {A}_{n - o ( λ 3 )} )}{log ( {A}_{n - o ( λ 1 )} )} - K$$

$${R}_{s 2 - o - 1} = \frac{log ( {A}_{s 2 - o ( λ 3 )} )}{log ( {A}_{s 2 - o ( λ 1 )} )} - K$$

$${R}_{s 1 - o - 2} = \frac{{A}_{s 1 - o ( λ 3 )}}{{A}_{s 1 - o ( λ 1 )}}$$

$${R}_{n - o - 2} = \frac{{A}_{s 1 - o ( λ 3 )}}{{A}_{s 1 - o ( λ 1 )}}$$

$${R}_{s 2 - o - 2} = \frac{{A}_{s 1 - o ( λ 3 )}}{{A}_{s 1 - o ( λ 1 )}}$$

Step S412: applying the first set of feature ratio values to the (n−1)th set of feature ratio values to a regression model to obtain a blood glucose value. Step S414: displaying the blood glucose value.

In one embodiment, if only two light sources are considered (corresponding to one group of feature ratio values), the processing unit 520, after calculating the six feature ratio values, applies these feature ratio values to the regression model to obtain the blood glucose value, which is then displayed on the display module 300. In another embodiment, if there are three light sources (corresponding to two groups of feature ratio values), the processing unit 520, after calculating the feature ratio values (a total of twelve feature ratio values), applies these feature ratio values to the regression model, and finally obtains a blood glucose value that may be different from applying only two light sources, and displays it on the display module 300.

Similarly, if the blood glucose meter 10 is equipped with a fourth light source, a fifth light source, and up to the nth light source, the processing unit 520 will calculate the difference values between the first set of difference values and up to the nth set of difference values. Subsequently, the processing unit 520 by using the aforementioned six equations will obtain six feature ratio values according to the first set of difference values and the second set of difference values, obtain another six feature ratio values according to the first set of difference values and the third set of difference values to, and so on, obtain yet another six feature ratio values (i.e., the (n−1)th group of feature ratio values which includes ((n−1)×2−1)th subgroup feature ratio values and ((n−1)×2)th subgroup feature ratio values) according to the first set of difference values and the nth set of difference values. Finally, the 6×(n−1) feature ratio values are applied to the regression model to obtain the blood glucose value.

In a preferred embodiment of the present disclosure, green light, red light, and near-infrared light are used as light sources for the blood glucose meter 10. Green light has a higher absorption rate for hemoglobin. This means that when the amount of hemoglobin in the blood increases or decreases, the amount of absorbed green light will correspondingly increase or decrease, reflecting changes in blood volume. Since blood glucose concentration affects the optical properties of blood, such as scattering and absorption characteristics, green light can be used to indirectly estimate blood glucose concentration by measuring changes in blood volume and other related parameters. Red light has a higher absorption rate for blood glucose (glucose). Therefore, when the red light passes through the blood, its scattering and absorption characteristics undergo slight changes due to variations in blood glucose concentration, and these changes can be detected to determine blood glucose concentration. Similarly, near-infrared light also has a high absorption rate for blood glucose (glucose). Thus, changes in near-infrared light passing through the blood can be used to determine variations in blood glucose concentration.

The blood glucose meter of the present disclosure has significant advantages. It uses two or more light sources to illuminate the skin to obtain more measurement data and employs specific algorithms to perform extensive calculations on the data to obtain more estimated blood glucose values for cross-referencing and calibration, thereby improving the accuracy of the final blood glucose value.

The above is only exemplary, rather than restrictive. Any equivalent modifications or changes without departing from the spirit and scope of the present disclosure should fall within the scope of the appended claims.

What is claimed is:

1. A blood glucose meter, comprising:
a first light source, having a first wavelength, configured to:
emit a first light energy towards human skin;
a second light source, having a second wavelength, configured to:
be positioned on the same side as the first light source and emit a second light energy towards human skin, wherein the second wavelength is greater than the first wavelength;
a light sensor, configured to:
be positioned on the opposite side of the first light source to convert the first light energy and the second light energy that have passed through human skin into a first electrical signal and a second electrical signal, respectively; or be positioned on the same side as the first light source to convert the first light energy and the second light energy reflected off human skin into the first electrical signal and the second electrical signal, respectively;
a PPG circuit, configured to:
drive the first light source and the second light source, and generate a first PPG response and a second PPG response based on the first electrical signal and the second electrical signal;
a processing unit, configured to execute the following steps:
performing feature extraction based on the first PPG response and the second PPG response to obtain a first set of feature values and a second set of feature values;
executing an algorithm based on the first set of feature values and the second set of feature values to obtain a first group of feature ratio values; and
applying the first group of feature ratio values to a regression model to obtain a blood glucose value; and
a display module, configured to:
display the blood glucose value.

2. The blood glucose meter according to claim 1, wherein the first group of feature ratio values includes a first subgroup of feature ratio values and a second subgroup of feature ratio values, and the algorithm includes the following operations:
performing a difference operation on the first set of feature values and the second set of feature values to obtain a first set of difference values and a second set of difference values;
performing logarithmic, division, and subtraction operations on the first set of difference values and the second set of difference values to obtain the first subgroup of feature ratio values; and
performing a division operation on the first set of difference values and the second set of difference values to obtain the second subgroup of feature ratio values.

3. The blood glucose meter according to claim 2, further comprising a third light source having a third wavelength, configured to: be positioned on the same side as the first light source and emit a third light energy towards human skin, wherein the third wavelength is greater than the first wavelength, wherein the light sensor is further configured to convert the third light energy that passed through human skin into a third electrical signal; or convert the third light energy reflected off human skin into the third electrical signal, wherein the PPG circuit is further configured to: drive the third light source and generate a third PPG response based on the third electrical signal, wherein, the processing unit is further configured to execute the following steps:
performing feature extraction based on the third PPG response to obtain a third set of feature values;
executing the algorithm based on the first set of feature values and the third set of feature values to obtain a second group of feature ratio values; and
applying the first group of feature ratio values and the second group of feature ratio values to the regression model to obtain the updated blood glucose value.

4. The blood glucose meter according to claim 3, wherein the second group of feature ratio values includes a third subgroup of feature ratio values and a fourth subgroup of feature ratio values, and the algorithm further includes the following operations:
performing a difference operation on the third set of feature values to obtain a third set of difference values;
performing logarithmic, division, and subtraction operations on the first set of difference values and the third set of difference values to obtain the third subgroup of feature ratio values; and
performing a division operation on the first set of difference values and the third set of difference values to obtain the fourth subgroup of feature ratio values.

5. The blood glucose meter according to claim 3, wherein the first light source is green light, the second light source is red light, and the third light source is near-infrared light.

6. A method for measuring blood glucose, comprising:
driving a first light source having a first wavelength and a second light source having a second wavelength by a PPG circuit, such that the first light source emits a first light energy towards human skin and the second light source emits a second light energy towards human skin, wherein the second wavelength is greater than the first wavelength;
converting the first light energy and the second light energy that have passed through human skin into a first electrical signal and a second electrical signal respectively, or converting the first light energy and the second light energy reflected off human skin into the first electrical signal and the second electrical signal respectively by a light sensor;
generating a first PPG response and a second PPG response based on the first electrical signal and the second electrical signal by the PPG circuit;
executing the following steps by a processing unit:
performing feature extraction based on the first PPG response and the second PPG response to obtain a first set of feature values and a second set of feature values;
executing an algorithm based on the first set of feature values and the second set of feature values to obtain a first group of feature ratio values; and
applying the first group of feature ratio values to a regression model to obtain a blood glucose value; and
displaying the blood glucose value by a display module.

7. The method according to claim 6, wherein the first group of feature ratio values includes a first subgroup of feature ratio values and a second subgroup of feature ratio values, and the algorithm includes the following operations:
performing a difference operation on the first set of feature values and the second set of feature values to obtain a first set of difference values and a second set of difference values;
performing logarithmic, division, and subtraction operations on the first set of difference values and the second set of difference values to obtain the first subgroup of feature ratio values; and
performing a division operation on the first set of difference values and the second set of difference values to obtain the second subgroup of feature ratio values.

8. The method according to claim 7, further comprising:
driving a third light source by the PPG circuit, such that the third light source emits a third light energy with a third wavelength towards human skin, wherein the third wavelength is greater than the first wavelength;
converting the third light energy that passed through human skin into a third electrical signal, or converting the third light energy reflected off human skin into the third electrical signal by the light sensor.
generating a third PPG response based on the third electrical signal by the PPG circuit; and
executing the following steps by the processing unit:
performing feature extraction based on the third PPG response to obtain a third set of feature values;
executing the algorithm based on the first set of feature values and the third set of feature values to obtain a second group of feature ratio values; and
applying the first group of feature ratio values and the second group of feature ratio values to the regression model to obtain the updated blood glucose value.

9. The method according to claim 8, wherein the second group of feature ratio values includes a third subgroup of feature ratio values and a fourth subgroup of feature ratio values, and the algorithm further includes the following operations:
performing a difference operation on the third set of feature values to obtain a third set of difference values;
performing logarithmic, division, and subtraction operations on the first set of difference values and the third set of difference values to obtain the third subgroup of feature ratio values; and
performing a division operation on the first set of difference values and the third set of difference values to obtain the fourth subgroup of feature ratio values.

10. The method according to claim 9, further comprising:
driving a nth light source by the PPG circuit, such that the nth light source emits an nth light source towards human skin, where the wavelength of the nth light source is greater than the first wavelength, and n>3;
converting the nth light energy that passed through the human skin into the nth electrical signal, or converting the nth light energy reflected off the human skin into the nth electrical signal by the light sensor;
generating an nth PPG response based on the nth electrical signal by the PPG circuit; and
executing the following steps by the processing unit:
performing feature extraction based on the nth PPG response to obtain the nth set of feature values;
performing a difference operation on the nth set of feature values to obtain the nth set of difference values;
performing logarithmic, division, and subtraction operations on the first set of difference values and the nth set of difference values to obtain a ((n−1)×2−1)th subgroup of feature ratio values;
performing a division operation on the first set of difference values and the nth set of difference values to obtain a ((n−1)×2)th subgroup of feature ratio values; and
applying the first subgroup of feature ratio values to the ((n−1)×2)th subgroup feature ratio values to the regression model to obtain the updated blood glucose value.
